(19) United States
(12) Patent Application Publication
(10) Pub. No.: US 2012/0065265 A1
(43) Pub. Date: Mar. 15, 2012

(54) ALPHA-2-DELTA LIGANDS FOR NON-RESTORATIVE SLEEP

(75) Inventors: TIMOTHY JAMES GRIFFIN, Saline, MI (US); Bruce Gerald McCarthy, Ann Arbor, MI (US); David Young Mitchell, Lafayette, CO (US); Daniele Marie-Claude Ouellet, Chapel Hill, NC (US); Theresa Papa Stern, Saline, MI (US); John Leroy Werth, JR., Pottstown, PA (US)

(73) Assignee: Pfizer Inc.

(21) Appl. No.: 13/297,769

(22) Filed: Nov. 16, 2011

Related U.S. Application Data
(63) Continuation of application No. 12/281,810, filed on Sep. 5, 2008.
(60) Provisional application No. 60/779,636, filed on Mar. 6, 2006.

(51) Int. Cl. A61K 31/197; A61P 25/00

(52) U.S. Cl. 514/561

(57) ABSTRACT

The use of an alpha-2-delta ligand or a pharmaceutically acceptable salt thereof for the treatment of non-restorative sleep is disclosed.

This application is a continuation of U.S. patent application Ser. No. 12/281,810, filed Sep. 5, 2008, which is a U.S. Nation Stage Application under 35 U.S.C. 371 of PCT/IB/2007/000458 filed Feb. 22, 2007, which in turn claims the benefit of priority to U.S. Provisional Patent Application Ser. No. 60/779,636 filed Mar. 6, 2006, the disclosure of each of which is hereby incorporated by reference in its entirety.

### BACKGROUND OF THE INVENTION

Problems with sleep are prevalent worldwide. See Roth, T. et al., Sleep Med 6:487-95 (2005). When describing sleep concerns, subjects usually complain of difficulty initiating sleep (DIS), of difficulty maintaining sleep (DMS), of awakening too early in the morning, or of a combination of these symptoms. However, a fourth complaint related to poor sleep has been described in the last several years as non-restorative sleep (NRS)—a feeling that the sleep episode has been un-refreshing or un-restoring, or what the DSM-IV describes as light, restless, or poor quality sleep. See American Psychiatric Association, Diagnostic and Statistical Manual of Mental Disorders (4th ed., Text Revision, 2000).

Complaints from subjects suffering from NRS include difficulty getting started in the morning, daytime fatigue, daytime sleepiness, general inability to function in the daytime, alertness problems, impaired mood, and poor work and academic performance. For those suffering from NRS, however, these complaints may not be the result of difficulties initiating or maintaining sleep. This was demonstrated in a multinational epidemiology study comprised of over 25,000 individuals, which showed that about 11% of the study population experienced NRS and that about 3% of the study population experienced NRS without the classic symptoms of DIS or DMS. See M. Ohayon, Arch Intern Med 165:35-41 (2005).

Alpha-2-delta (α2δ) ligands are known to bind the α2δ subunits of calcium channels. Published U.S. Patent Application No. 2005/0059654 describes methods for treating depression in mammals, as well as depression and a concomitant disease, including anxiety, sleep disorder and post-traumatic stress disorder, comprising administering various combinations of an α2δ ligand with a serotonin re-uptake inhibitor (SSRI) or with a selective noradrenaline re-uptake inhibitor (SNRI), or both.

Published U.S. Patent Application No. 2004/0092522 describes combinations of an α2δ ligand and a cyclic guanosine 3′,5′-monophosphate phosphodiesterase type 5 (PDEV) inhibitor for use in treating pain.

Published U.S. Patent Application No. 2004/0180959 describes the use of cyclic α2δ ligands for treating fibromyalgia or fibromyalgia and a concomitant disorder, and their use in combination with a human growth hormone or human growth hormone secretagogue for increasing slow wave sleep. Published U.S. Patent Application No. 2004/0186177 describes the use of acyclic α2δ ligands for treating various disorders, including fibromyalgia and hot flashes.

Published U.S. Patent Application Nos. 2003/0195251 and 2005/0124668 describe β-amino acids that bind to the α2δ subunit of calcium channels and are useful for treating central nervous system disorders.

Published U.S. Patent Application No. 2003/0212133 describes the use of cyclic α2δ ligands for treating insomnia.

### SUMMARY OF THE INVENTION

This invention relates to methods for treating non-restorative sleep (NRS). For the purposes of this invention, NRS is defined as awakening un-refreshed or un-restored. These symptoms are not due to difficulty initiating sleep, difficulty maintaining sleep, or awakening too early. NRS does not occur exclusively during the course of another sleep disorder or mental disorder, and is not due to direct physiological effects of a substance or a general medical condition. To be diagnosed with NRS a subject or patient: (a) exhibits clinically significant distress or impairment in social, occupational or other areas of daytime functioning; (b) does not report (either subjectively or objectively by polysomnography) difficulty initiating sleep (DIS) or difficulty maintaining sleep (DMS); and (c) exhibits the symptoms in (a) at least 3 times/week for a period of at least 1 month.

This invention provides a method of treating non-restorative sleep in a subject in need of such treatment. The method comprises administering a therapeutically effective amount of a compound, or a pharmaceutically acceptable salt thereof, in which the compound (or its salt) is an α2δ ligand.

One aspect of the invention provides that the compound is a γ-amino acid or a pharmaceutically acceptable salt thereof.

Another aspect of the invention provides that the compound is gabapentin or a pharmaceutically acceptable salt thereof.

Another aspect of the invention provides that the compound is pregabalin or a pharmaceutically acceptable salt thereof.

Another aspect of the invention provides that the α2δ ligand is a compound of formula 1 or 1A,

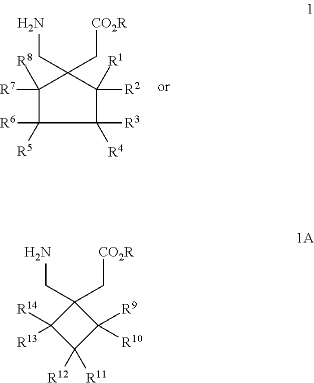

or a pharmaceutically acceptable salt thereof wherein:

R is hydrogen or a straight or branched alkyl having from 1 to 4 carbon atoms; and

R¹ to R¹⁴ are each independently selected from hydrogen, straight or branched alkyl of from 1 to 6 carbon atoms, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, or —OR¹⁵, wherein R¹⁵ is a straight or branched alkyl of from 1 to 6 carbon atoms, phenyl, or benzyl, and R¹ to R⁸ are not simultaneously hydrogen.

Another aspect of the invention provides that the compound is (3S,4S)-(1-aminomethyl-3,4-dimethyl-cyclopentyl)-acetic acid or a pharmaceutically acceptable salt thereof.

Another aspect of the invention provides that the compound is a β-amino acid or a pharmaceutically acceptable salt thereof.

This invention is also provides a method for treating non-restorative sleep in a subject in need of treatment, the method comprising:

diagnosing the subject having non-restorative sleep, and

administering to the subject &therapeutically effective amount of a compound, or a pharmaceutically acceptable salt thereof, in which the compound (or its salt) is an α2δ ligand.

One aspect of the invention is that the compound is selected from (3S,4S)-(1-aminomethyl-3,4-dimethyl-cyclopentyl)-acetic acid, gabapentin, and pregabalin, or a pharmaceutically salt of the foregoing compounds.

Useful compounds are α2δ ligands, and include compounds described in published United States Patent Application Nos. 2005/0059654, 2004/0092522, 2004/0180959, 2004/0186177, 2003/0195251, 2005/0124668, and 2003/0212133, as well as published International Patent Application No. WO 04,054,566.

Useful compounds include the α2δ ligands (3S,4S)-(1-aminomethyl-3,4-dimethyl-cyclopentyl)-acetic acid, (1-aminomethyl-cyclohexyl)-acetic acid (gabapentin), and the S-(+) enantiomer of 4-amino-3-(2-methylpropyl) butanoic acid (pregabalin).

Methods for determining whether a particular compound is an α2δ ligand (i.e. whether a particular compound binds the α2δ subunit of a calcium channel) include those described in N. S. Gee et al., J. Biol. Chem. 271:5768-5776 (1996); E. Marais et al., Mol. Pharmacol. 59:1243-1248 (2001); H. C. Gong et al., J. Membr. Biol. 184:35-43 (2001); and N. Qin et al., Mol. Pharmacol. 62:485-496 (2002). Useful compounds generally exhibit an IC₅₀ (concentration at 50% inhibition) of about 1 μM or less or about 0.5 μM or less.

Useful compounds include all pharmaceutically acceptable complexes, salts, solvates, and hydrates thereof, as well as all stereoisomers, tautomers, and polymorphic forms thereof, including all crystalline and amorphous forms, whether they are pure, substantially pure, or mixtures. Useful compounds may also be combined with other agents, including agents that enhance sleep inducing effects. Such agents include melatonin, tryptophan, valerian, passiflora, antihistamines, such as diphenydramine hydrochloride or doxylamine succinate, benzodiazepines, and non-benzodiazepine hypnotics.

### DETAILED DESCRIPTION OF THE INVENTION

Unless otherwise indicated, this disclosure uses definitions provided below. Some of the definitions and formulae may include a dash (“-”) to indicate a bond between atoms or a point of attachment to a named or unnamed atom or group of atoms.

“Substituted” groups are those in which one or more hydrogen atoms have been replaced with one or more non-hydrogen atoms or groups, provided that valence requirements are met and that a chemically stable compound results from the substitution.

“About” or “approximately,” when used in connection with a measurable numerical variable, refers to the indicated value of the variable and to all values of the variable that are within the experimental error of the indicated value (e.g., within the 95% confidence interval for the mean) or within ±10 percent of the indicated value, whichever is greater.

“Alkyl” refers to straight or branched hydrocarbon groups having from 1 to 6 carbon atoms and includes methyl, ethyl, propyl, isopropyl, butyl, 2-butyl, tert-butyl, and pentyl.

“Alkoxy” refers to alkyl-O—, where alkyl is defined above, and includes methoxy, ethoxy, propoxy, isopropoxy, butoxy, 2-butoxy, tert-butoxy, and pentyloxy.

“Carboalkoxy” refers to alkoxy-C(O)—, where alkoxy is defined above, and includes methoxycarbonyl, ethoxycarbonyl, isopropoxycarbonyl, butoxycarbonyl, 2-butoxycarbonyl, tert-butoxycarbonyl, and pentyloxycarbonyl.

“γ-amino acid” refers to a compound having a monovalent or divalent radical selected from (4-amino-butanoic acid)-3-yl and (4-amino-butanoic acid)-3,3-diyl, respectively.

“β-amino acid” refers to a compound having a monovalent radical selected from (3-amino-propanoic acid)-2-yl and (3-amino-propanoic acid)-3-yl.

Benzyl and phenyl groups may be unsubstituted or substituted with from 1 to 3 substituents selected from hydroxy, carboxy, carboalkoxy, halogen, —CF₃, nitro, alkyl, and alkoxy. Preferred substituents include one or more halogens.

“Subject” refers to a mammal, including a human.

“Pharmaceutically acceptable” substances refers to those substances which are within the scope of sound medical judgment suitable for use in contact with the tissues of subjects without undue toxicity, irritation, allergic response, and the like, commensurate with a reasonable benefit-to-risk ratio, and effective for their intended use.

“Treating” refers to reversing, alleviating, inhibiting the progress of, or preventing a disorder or condition to which such term applies, or to reversing, alleviating, inhibiting the progress of, or preventing one or more symptoms of such disorder or condition.

“Treatment” refers to the act of “treating,” as defined immediately above. “Drug,” “drug substance,” “active pharmaceutical ingredient,” and the like, refer to a compound (e.g., compounds of formula 1 and formula 1A, and compounds specifically named above) that may be used for treating a subject in need of treatment.

“Therapeutically effective amount” of a drug refers to the quantity of the drug that may be used for treating a subject and may depend on the weight and age of the subject and the route of administration, among other things.

“Inert” substances refer to those substances that may influence the bioavailability of the drug, but are otherwise pharmacologically inactive.

“Excipient” or “adjuvant” refers to any inert substance.

“Pharmaceutical composition” refers to the combination of one or more drug substances and one or more excipients.

“Drug product,” “pharmaceutical dosage form,” “dosage form,” “final dosage form” and the like, refer to a pharmaceutical composition that is administered to a subject in need of treatment and generally may be in the form of tablets, capsules, sachets containing powder or granules, liquid solutions or suspensions, patches, films, and the like.

Many of the compounds which are useful for treating NRS, including compounds represented by formula 1, formula 1A, and compounds specifically named above, may form pharmaceutically acceptable complexes, salts, solvates and hydrates. These salts include acid addition salts (including di-acids) and base salts. Pharmaceutically acceptable acid addition salts include nontoxic salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include nontoxic salts derived from bases, including metal cations, such as an alkali or alkaline earth metal cation, as well as amines. Examples of suitable metal cations include sodium (Na⁺), potassium (K⁺), magnesium (Mg²⁺), calcium (Ca²⁺), zinc (Zn²⁺), and aluminum (Al³⁺). Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., “Pharmaceutical Salts,” 66 J. Pharm. Sci. 1-19 (1977); see also Stahl and Wermuth, Handbook of Pharmaceutical Salts: Properties, Selection, and Use (2002).

Pharmaceutically acceptable salts may be prepared using various methods. For example, one may react a compound of formula 1 with an appropriate acid or base to give the desired salt. One may also react a precursor of the compound of formula 1 with an acid or base to remove an acid- or base-labile protecting group or to open a lactone or lactam group of the precursor. Additionally, one may convert a salt of the compound of formula 1 to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

The compounds used to treat NRS may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

The compounds used to treat NRS may also exist as multi-component complexes (other than salts and solvates) in which the compound (drug) and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun. 17:1889-1896 (2004). For a general review of multi-component complexes, see J. K. Haleblian, J. Pharm. Sci. 64(8):1269-88 (1975). All references to compounds, including compounds of formula 1, formula 1A, and compounds described and named in the specification, generally include all polymorphs and crystal habits, prodrugs, metabolites, stereoisomers, and tautomers thereof, as well as all isotopically-labeled compounds thereof.

“Prodrugs” refer to compounds having little or no pharmacological activity that can, when metabolized in vivo, undergo conversion to compounds having desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester or amide derivatives of compounds of formula 1, formula 1A, and compounds described and named in the specification, having carboxylic acid or amino functional groups, respectively. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds of formula 1, formula 1A, and compounds described and named in the specification, having methyl, alkoxy, tertiary amino, secondary amino, phenyl, and amide groups, respectively.

Certain compounds described herein may have stereoisomers. Some of these compounds may exist as single enantiomers (enantiopure compounds) or mixtures of enantiomers (enriched and racemic samples), which depending on the relative excess of one enantiomer over another in a sample, may exhibit optical activity. Such stereoisomers, which are non-superimposable mirror images, possess a stereogenic axis or one or more stereogenic centers (i.e., chirality). Other compounds may be stereoisomers that are not mirror images. Such stereoisomers, which are known as diastereoisomers, may be chiral or achiral (contain no stereogenic centers). They include molecules containing an alkenyl or cyclic group, so that cis/trans (or Z/E) stereoisomers are possible, or molecules containing two or more stereogenic centers, in which inversion of a single stereogenic center generates a corresponding diastereoisomer. Unless stated or otherwise clear (e.g., through use of stereobonds, stereocenter descriptors, etc.) the scope of the invention and disclosure generally includes the reference compound and its stereoisomers, whether they are each pure (e.g., enantiopure) or mixtures (e.g., enantiomerically enriched or racemic). “Tautomers° refer to structural isomers that are interconvertible via a low energy barrier.

Tautomeric isomerism (tautomerism) may take the form of proton tautomerism in which the compound contains, for example, an imino, keto, or oxime group, or valence tautomerism in which the compound contains an aromatic moiety.

Compounds described herein also include all pharmaceutically acceptable isotopic variations, in which at least one atom is replaced by an atom having the same atomic number, but an atomic mass different from the atomic mass usually found in nature. Isotopes suitable for inclusion in compounds include, for example, isotopes of hydrogen, such as ²H and ³H; isotopes of carbon, such as¹¹C, ¹³C and ¹⁴C; isotopes of nitrogen, such as¹³N and ¹⁵N; isotopes of oxygen, such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labeled compounds may be prepared by processes analogous to those described elsewhere in the disclosure using an appropriate isotopically-labeled reagent in place of a non-labeled reagent.

Compounds of formula 1, formula 1A, and compounds described and named above, and their pharmaceutically acceptable complexes, salts, solvates and hydrates, should be assessed for their biopharmaceutical properties, such as solubility and solution stability across pH, permeability, and the like, to select an appropriate dosage form and route of administration. Compounds that are intended for pharmaceutical use may be administered as crystalline or amorphous products, and may be obtained, for example, as solid plugs, powders, or films by methods such as precipitation, crystallization, freeze drying, spray drying, evaporative drying, microwave drying, or radio frequency drying.

The compounds used to treat NRS can be prepared and administered in a wide variety of oral and parenteral dosage forms. Thus, the compounds can be administered by injection, i.e., intravenously, intramuscularly, intracutaneously, subcutaneously, intraduodenally, or intraperitoneally. Also, the compounds of the present invention can be administered by inhalation, for example, intranasally. Additionally, the compounds can be administered transdermally. When describing dosage forms, the active pharmaceutical ingredient refers to the compounds of formula 1, formula 1A, and compounds described and named in specification as well as their pharmaceutically acceptable complexes, salts, solvates and hydrates.

In addition to the active pharmaceutical ingredient (API), pharmaceutical compositions include pharmaceutically acceptable carrier that can be either solid or liquid. Solid dosage forms include powders, tablets, pills, capsules, cachets, suppositories, and dispersible granules. A solid carrier is generally inert and may comprise one or more substances (excipients) which may also act, for example, as diluents, flavoring agents, binders, preservatives, tablet disintegrating agents, or an encapsulating material. For powders, the carrier is a finely divided solid which is in a mixture with the finely divided API; for tablets, the API is typically mixed with the carrier having the necessary binding properties in suitable proportions and compacted in the shape and size desired.

Powders and tablets generally contain from about 5% to about 70% of the API based on weight. Suitable excipients include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” may include the formulation of the API with encapsulating material as a carrier that provides a capsule in which the active component, with or without other carriers, is surrounded by a carrier, which is thus in association with it. Tablets, powders, capsules, cachets, and lozenges can be used as solid dosage forms suitable for oral administration.

For preparing suppositories, a low melting wax, such as a mixture of fatty acid glycerides or cocoa butter, is first melted, and the API is dispersed homogeneously therein, as by stirring. The molten homogenous mixture is then poured into convenient sized molds, allowed to cool, and thereby to solidify.

Liquid pharmaceutical compositions include solutions, suspensions, and emulsions, which comprise, for example, water or aqueous propylene glycol solutions. Liquid preparations suitable for parenteral injection may be formulated in aqueous polyethylene glycol solution.

Aqueous solutions suitable for oral use can be prepared by dissolving the API in water and adding suitable colorants, flavors, stabilizing and thickening agents as desired. Aqueous suspensions can be made by dispersing finely divided API in water with viscous material, such as natural or synthetic gums, resins, methylcellulose, sodium carboxymethylcellulose, and other suspending agents.

Useful pharmaceutical compositions also include solid preparations which are intended to be converted, shortly before use, to liquid pharmaceutical compositions suitable oral administration. Such liquid dosage forms include solutions, suspensions, and emulsions. These preparations may contain, in addition to the API, colorants, flavors, stabilizers, buffers, artificial and natural sweeteners, dispersants, thickeners, solubilizing agents, and the like.

The pharmaceutical composition is preferably in unit dosage form. In such cases, the pharmaceutical composition is subdivided into unit doses containing appropriate quantities of the API. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1 g according to the particular application and the potency of the active component. To treat

NRS, the drug is typically administered once daily before bedtime, as for example, capsules or tablets containing of 1 mg, 5 mg, 10 mg, 25 mg, 50 mg, 75 mg, 100 mg, 200 mg, 300 mg, 400 mg or 500 mg of the API. The composition may, if desired, also contain other compatible therapeutic agents.

For therapeutic use, the compounds utilized in the disclosed method may be administered at an initial dosage of about 0.01 mg/kg daily to about 100 mg/kg daily. A daily dose range of about 0.02 mg/kg to about 10 mg/kg is typical. The dosages, however, may be varied depending upon the requirements of the patient, the severity of the condition being treated, and the compound being employed. Determination of the proper dosage for a particular situation is within the skill of the art. In some cases, treatment is initiated with smaller dosages which are less than the optimum dose of the compound. Thereafter, the dosage is increased by small increments until the optimum effect under the circumstances is reached.

### EXAMPLE

The following example is intended to be illustrative and non-limiting and represents a specific embodiment of the present invention. The term “COMPOUND A” refers to the a26 ligand (3S,4S)-(1-aminomethyl-3,4-dimethyl-cyclopentyl)-acetic acid.

### Methodology

STUDY DESIGN. A randomized, double-blind, placebo- and active-controlled, multicenter, 4-way crossover study was carried out to evaluate the effects of COMPOUND A in an NRS population. Subjects with NRS who met screening requirements were initially randomized into the study and received 1 of 4 treatments (COMPOUND A 25 mg, COMPOUND A 50 mg, zolpidem 10 mg, or placebo) daily for 2 weeks followed by a 1-week washout period. Subjects were subsequently crossed over 3 times to receive the remaining treatments, each for 2 weeks followed by a 1—week washout period. Each subject was orally dosed (blinded capsule), either 1 hour before bedtime with COMPOUND A or placebo, or 30 minutes before bedtime with zolpidem or placebo. Each treatment was administered at least 2 hours after a meal. DIAGNOSIS AND MAIN CRITERIA FOR INCLUSION. Subjects selected for the study were male and/or nonpregnant, nonlactating females between the ages of 18-64 years, inclusive, who regularly (≧3 times/week) awoke un-restored or un-refreshed for at least the previous 3 months, causing significant distress or impairment in social, occupational, or other important areas of functioning during the daytime.

EFFICACY & SAFETY EVALUATIONS. The primary endpoint in this study was the Restorative Sleep Questionnaire—Weekly (RSQ-W) Total Score at the end of each 2-week treatment period. The RSQ-W, below, is a patient-reported outcome measure of morning refreshment over the past week in which larger values of the total score correspond to greater refreshment. All remaining efficacy/outcomes endpoints were considered secondary. Safety data, including adverse event information, clinical laboratory values, physical examination, vital signs, and electrocardiograms (ECGs) were collected during the study.

STATISTICAL METHODS. The Full Analysis Set was used in the analyses of all efficacy/outcome endpoints. The Full Analysis Set consisted of all randomized subjects who took any study medication and who had a baseline with at least 1 post-baseline measurement on at least 1 efficacy/outcome variable. The Per-Protocol Analysis Set was used for sensitivity analyses of certain efficacy/outcome endpoints as appropriate. The Per-Protocol Analysis set included all subjects from the Full Analysis Set who did not have major protocol deviations. Protocol deviations included the major inclusion/exclusion criteria assessed prior to randomization and major protocol deviations or violations assessed after randomization. Partial data for subjects who took incorrect treatment (as a protocol deviation assessed after randomization) may have been included in the Per-Protocol Analysis Set. The Safety Analysis Set was used in the analyses of the safety data and consisted of all randomized subjects who took any study medication.

For the primary efficacy endpoint, each active treatment was compared to placebo. For each active treatment, the null hypothesis tested was that there is no difference in the true means for this endpoint between the active treatment and placebo. The corresponding alternative hypothesis was that there is a difference in the true means for this endpoint in favor of the active treatment compared to placebo. Each comparison was done at the nominal alpha=0.05 level (one-sided), recognizing that the probability of committing at least one Type I error could be greater than 0.05 but no more than 0.15.

MODEL-BASED SUMMARY. The primary endpoint in this study was the RSQ-W Total Score at the end of each 2-week treatment period. This endpoint was analyzed using a linear model including sequence, period, and treatment as fixed factors, and subject within sequence and within-subject error as random factors. First-order carryover effects were explored and tested at the 10% nominal level of significance. Pair-wise comparisons were made based on the final linear model. The point estimates and 90% confidence intervals (Cis) for the placebo-adjusted treatment effects were constructed using the least squares (LS) means and appropriate standard errors.

DESCRIPTIVE SUMMARY. For each item and Total Score of the RSQ-W, descriptive statistics were provided by treatment and visit.

For secondary/exploratory purposes, certain secondary efficacy endpoints were analyzed with model-based statistical procedures. For each of these endpoints, the null hypothesis was that there is no difference in the true means between the active treatment and placebo. The corresponding alternative hypothesis was that there is a difference in the true means in favor of the active treatment compared to placebo, with the understanding that the direction of the one-sided alternative hypothesis depends on the direction of the endpoint being tested (i.e., the direction of the one-sided alternative hypothesis is endpoint-specific). All comparisons are considered secondary/exploratory, and each was done at the nominal alpha=0.05 level (one-sided). No multiple comparison adjustment was made. All secondary efficacy endpoints were summarized descriptively. For secondary/exploratory purposes, appropriate model-based statistical procedures similar to those for the primary endpoint were used to analyze the following scales and subscales:
Restorative Sleep Questionnaire—Daily (RSQ-D, below): weekly averages of Total Score;
Daytime Consequences of Sleep Questionnaire (DCSQ): Total Score;
Multidimensional Assessment of Fatigue (MAF): Global Fatigue Index, Impact Subscale;
Subjective Sleep Questionnaire (SSQ): Sleep Quality;
Sheehan Disability Scale (SDS): Total Score;
SF-36v2: Mental Component Summary, Physical Component Summary, Vitality Subscale;
Clinical Global Impression of Change (CGIC): Status Score; and
Patient Global Impression of Change (PGIC): Status Score.

### Results

SUBJECT DISPOSITION AND DEMOGRAPHY. A total of 149 subjects were screened and 58 subjects were assigned to treatment. Of these, COMPOUND A 50 mg and zolpidem treatments were completed by 51 subjects each and COMPOUND A 25 mg and placebo treatments were completed by 50 subjects each. A total of 9 subjects discontinued from the study.

EFFICACY RESULTS. With regards to the primary efficacy parameter, the estimated mean (LS Mean) value for the RSQ-W Total Score for the COMPOUND A 25 mg dose group (63.6) was statistically significantly different from placebo (58.8) and zolpidem (57.7), and the LS Mean for the COMPOUND A 50 mg dose group (62.5) was statistically significantly different from zolpidem. Zolpidem was not statistically significantly different from placebo and COMPOUND A 50 mg was not statistically significantly different from the 25 mg dose.

For the secondary efficacy parameters, RSQ-D Total Scores at Week 2 showed no statistically significant differences from placebo for the COMPOUND A doses. The COMPOUND A 25 mg dose (61.5) was statistically significantly different from zolpidem (57.7). DCSQ scores (ranging from 85.7 to 87.6, on a scale of 0 to 100) were indicative of good daytime functioning for all of the treatment groups; none of the treatments were statistically significantly different from placebo. For the MAF indices at Week 2, the Impact Subscale scores were low (indicating a low impact from fatigue) and equivalent across all treatment groups; the COMPOUND A doses were not statistically significantly different from placebo. The Global Fatigue Index scores were low in all treatment groups, with the lowest LS means observed in the COMPOUND A 25 mg and 50 mg groups (11.3 and 12.4, respectively), but not significantly different from placebo (13.4). The SSQ scores were statistically significantly higher for the COMPOUND A 25 mg dose (LS Mean of 76.8) compared with placebo (LS Mean of 73.3) and SDS Total Score at Week 2 was statistically significantly lower (indicating less disability) for the COMPOUND A 25 mg group (3.4) compared with placebo (4.5). No statistically significant differences from placebo were observed for the Mental and Physical Component Summaries of the SF-36v2, but a significant difference from placebo (60.2) in the measurement of Vitality was observed for the COMPOUND A 25 mg dose group (66.0). No statistically significant differences between the COMPOUND A 25 mg and 50 mg doses (3.0 and 3.1, respectively) and placebo (3.3) were observed in the CGIC at Week 2. Based on the PGIC scores, all treatment groups reported minimal improvement with no significant treatment differences. With regards to the Evening Functioning Scale, subjects in the COMPOUND A dose groups generally reported better functioning than while taking placebo and a similar level of functioning as when taking zolpidem.

### CONCLUSIONS

The a2δ ligand, (3S,4S)-(1-aminomethyl-3,4-dimethyl-cyclopentyl)-acetic acid, demonstrated a positive treatment effect relative to placebo in subjects with non-restorative sleep on the primary endpoint, the RSQ-W. Zolpidem was not differentiated from placebo. The findings on the primary endpoint are supported by similar results on the secondary efficacy parameters. All of the treatments were well tolerated; no serious adverse events were reported and no clinically significant changes from screening were observed for laboratory values, vital sign measurements, or ECG results.

As used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. All numerical ranges described herein having one or more endpoints include the endpoints and all numerical values between the endpoints. The above description is intended to be illustrative and not restrictive. Many embodiments will be apparent to those of skill in the art upon reading the above description. Therefore, the scope of the invention should be determined with reference to the appended claims and includes the full scope of equivalents to which such claims are entitled. The disclosures of all articles and references, including patents, patent applications and publications, are herein incorporated by reference in their entirety and for all purposes.

1. A method for treating a subject suffering from non-restorative sleep, the method comprising administering a therapeutically effective amount of a compound or a pharmaceutically acceptable salt thereof, the compound being an alpha-2-delta ligand.

2. The method according to claim 1, wherein the compound is a γ-amino acid or a pharmaceutically acceptable salt thereof.

3. The method according to claim 1, wherein the compound is gabapentin or a pharmaceutically acceptable salt thereof.

4. The method according to claim 1, wherein the compound is pregabalin or a pharmaceutically acceptable salt thereof.

5. The method according to claim 1, wherein the compound is represented by formula 1 or formula 1A, 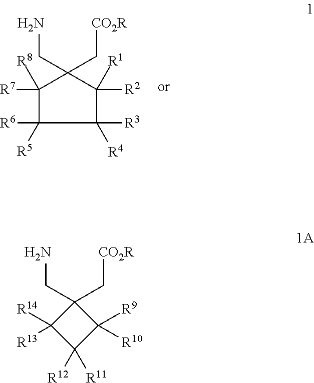
or is a pharmaceutically acceptable salt thereof, wherein:
R is hydrogen or a straight or branched alkyl of from 1 to 4 carbon atoms;
R¹ to R¹⁴ are each independently selected from hydrogen, straight or branched alkyl of from 1 to 6 carbon atoms, phenyl, benzyl, fluorine, chlorine, bromine, hydroxy, hydroxymethyl, amino, aminomethyl, trifluoromethyl, —CO₂H, —CO₂R¹⁵, —CH₂CO₂H, —CH₂CO₂R¹⁵, —OR¹⁵, wherein R¹⁵ is a straight or branched alkyl of from 1 to 6 carbon atoms, phenyl, or benzyl, and R¹ to R⁸ are not simultaneously hydrogen.

6. The method according to claim 1, wherein the compound is (3S,4S)-(1-aminomethyl-3,4-dimethyl-cyclopentyl)-acetic acid or a pharmaceutically acceptable salt thereof.

7. The method according to claim 1, wherein the compound is a p-amino acid or a pharmaceutically acceptable salt thereof.

8. The method according to claim 1, the method further comprising diagnosing the subject with non-restorative sleep.

9. The method according to claim 2, the method further comprising diagnosing the subject with non-restorative sleep.

10. The method according to claim 3, the method further comprising diagnosing the subject with non-restorative sleep.

11. The method according to claim 4, the method further comprising diagnosing the subject with non-restorative sleep.

12. The method according to claim 5, the method further comprising diagnosing the subject with non-restorative sleep.

13. The method according to claim 6, the method further comprising diagnosing the subject with non-restorative sleep.

14. The method according to claim 7, the method further comprising diagnosing the subject with non-restorative sleep.
